(19) United States
(12) Patent Application Publication
(10) Pub. No.: US 2016/0361394 A1
(43) Pub. Date: Dec. 15, 2016

(54) TREATMENT OF HEREDITARY ANGIOEDEMA WITH C1 INHIBITOR

(71) Applicant: SANTARUS, INC., Raleigh, NC (US)

(72) Inventor: Mark C. Totoritis, Rancho Santa Fe, CA (US)

(73) Assignee: Santarus, Inc., Raleigh, NC (US)

(21) Appl. No.: 15/122,130

(22) PCT Filed: Feb. 27, 2015
(86) PCT No.: PCT/US2015/018187

§ 371 Date: Aug. 26, 2016

Related U.S. Application Data
(60) Provisional application No. 61/946,677, filed on Feb. 28, 2014.

(51) Int. Cl. A61K 38/57; A61K 9/00

(52) U.S. Cl. CPC A61K 38/57; A61K 9/0019

(57) ABSTRACT

A method for treating acute attacks of hereditary angioedema (HAE) whereby a first does and a second dose of a recombinant C1 esterase inhibitor is administered intravenously to the patient, each dose at 50 IU/kg body weight of the patient and wherein the first and second doses are administered within a 24 hour period. The recombinant C1 esterase inhibitor has an amino acid sequence identical to the amino acid sequence of human plasma-derived C1 esterase inhibitor and a modified carbohydrate structure as compared to the human plasma-derived C1 esterase inhibitor. Relief of attack symptoms as well as reduction of relapse and/or new attack symptoms are achieved by use of the method.

### CROSS REFERENCE TO RELATED APPLICATIONS

This application claims benefit under 35 USC 119(e) to U.S. Provisional Application No. 61/946,677, filed Feb. 28, 2014, which is incorporated herein by reference in its entirety for all purposes.

### BACKGROUND

Human C1 inhibitor (C1INH), also known as C1 esterase inhibitor, is a substance in the superfamily of serine proteinase inhibitors. The production of recombinant C1 inhibitor in the milk of a transgenic nonhuman mammal (rhC1INH) is disclosed in U.S. Pat. No. 7,067,713. That patent also indicates that the C1 inhibitor (or its recombinant preparation) is useful in treating patients with HAE or patients requiring immunosuppression.

HAE is a rare and potentially life-threatening disorder that is understood to be an autosomal-dominant genetic disorder. Hereditary angioedema with C1 esterase inhibitor deficiency is characterized by recurrent attacks of tissue swelling. For example, HAE attacks can present as recurrent episodes of facial, peripheral, pharynegeal/laryngeal, gastrointestinal (GI) tract/abdominal or urogenital swelling. Patients suffering from HAE attacks can also suffer severe pain, disability, distension, nausea, etc., and may require hospitalization or experience a disruption of school, work and social interactions and sleep. Acute attacks are unpredictable and often occur without an apparent trigger. There is a need in the art for methods of achieving relief of symptoms for patients with acute HAE attacks.

### SUMMARY

The present invention is based, in part, on the discovery of a novel method of treating acute attacks of HAE in human patients. The methods and dosing regimens described herein may be advantageous for and, in some instances, critical to the survival of patients suffering from HAE. Groups of patients that could benefit from the dosing regimens include, without exclusion: individuals with life-threatening HAE symptoms, patients who do not experience relief from symptoms of HAE attacks by 4 hours after a first dose of a recombinant C1 inhibitor, and patients who only experience limited relief (less than 20 mm decrease in VAS score) after a first dose of a recombinant C1 inhibitor.

One aspect described herein is a method for treating an acute attack of hereditary angioedema (HAE) in a patient. The method includes administering intravenously to the patient a first dose of a recombinant C1 esterase inhibitor at 50 IU/kg body weight of the patient then administering intravenously to the patient a second dose of the recombinant C1 esterase inhibitor at 50 IU/kg body weight of the patient after administration of the first dose, thereby treating the acute attack of HAE in the patient.

In some embodiments, the first dose is administered within five hours from onset of the HAE attack in the patient. In further embodiments, the second dose is administered at least four hours after the first dose. In still further embodiments, the first dose and the second dose are administered within a 24 hour period. In yet further embodiments, no more than two doses are administered within a 24 hour period.

In some embodiments, the method is practiced in patient having multiple HAE attack sites. The attack site may be peripheral, abdominal, facial, oropharyngeal, and/or laryngeal. In further embodiments, the HAE attack is manifested in the form of life-threatening symptoms in the patient. In still further embodiments, the attack may have a severity rating of at least 50 mm on a Visual Analog Scale (VAS) of 100 mm.

In some embodiments, the method is practiced in patients in whom the beginning of relief of symptoms occurs within 4 hours from the first dose and the extent of the relief is less than 20 mm decrease in VAS score prior to the second dose. The decrease in VASS score may, in some instances, be measured based on two consecutive time points.

In some embodiments, the method is practiced in patients in whom HAE attack symptoms persist after the first dose and prior to administration of the second dose.

In some embodiments, the recombinant C1 inhibitor described herein has an amino acid sequence identical to the amino acid sequence of human plasma-derived C1 esterase inhibitor and a modified carbohydrate structure as compared to the human plasma-derived C1 esterase inhibitor. In further embodiments, the recombinant C1 inhibitor is purified from the milk of transgenic mammals. In still further embodiments, the recombinant C1 inhibitor is rhC1HNH.

In any of the embodiments described herein, the first and second doses of recombinant C1 esterase inhibitor may be self-administered by the patient.

In another aspect, provided herein, is a method of treating HAE, wherein the method involves administering a composition comprising a C1 inhibitor wherein substantial relief of symptoms is achieved within 4 hours or less. Another aspect described herein is a method for treating a patient suffering from an acute HAE attack comprising administering to the patient a composition comprising a C1 inhibitor wherein the treatment substantially relieves the patient of symptoms from the acute HAE attack and there is no recurrence of symptoms within 12 hours, or preferably 24 hours, or more preferably 48 hours, or further preferably 72 hours. Moreover, there is preferably no new acute HAE attack within 12 hours, more preferably 24 hours, or more preferably 48 hours, or further preferably 72 hours.

In another aspect described herein is a method for treating a patient suffering from HAE comprising administering to the patient a composition comprising a C1 inhibitor wherein the treatment provides substantial relief of HAE symptoms but does not substantially elevate the patient D-dimer level. More preferably, the D-dimer level is not substantially elevated over a period of at least 7 days from the administration of the C1 inhibitor composition.

In a further embodiment is a method for treating a patient suffering from HAE comprising administering to the patient a composition comprising a C1 inhibitor wherein the treatment provides substantial relief of HAE symptoms but does not substantially increase the risk of a thromboembolic event. The treatment preferably does not substantially increase the risk of deep vein thrombosis.

For each of the above embodiments, the C1 inhibitor composition can be administered to a patient with one or more submucosal or subcutaneous locations of attack. In a preferred embodiment, the C1 inhibitor is a recombinant C1 inhibitor such as rhC1HNH. In each of the above aspects, the composition is administered as a single dose or multiple doses, preferably in a single dose. The composition is administered in a dosage of 25 to 100 IU/kg, more preferably at about 50 IU/kg, and most preferably at 50 IU/kg.

In the above embodiments, the C1 inhibitor composition is preferably administered to provide relief of HAE symptoms. In a preferred embodiment, one of those symptoms is tissue swelling due to HAE. The C1 inhibitor composition can be administered to a patient suffering from HAE or an acute HAE attack. Other aspects and embodiments are described infra.

### BRIEF DESCRIPTION OF THE DRAWINGS

FIG. 1 is a pie graph showing a breakdown of HAE attacks according to the number of rhC1INH doses administered (single dose vs. two doses) to patients enrolled in the studies of Example 1. A single dose of rhC1INH was administered in 93% of attacks whereas two doses of rhC1INH were administered in 7% of attacks.

FIG. 2 is a chart depicting the response and relapse rates in the rhC1INH-treated patients for the studies described in Example 1. For these studies, a response was defined as the beginning of relief of symptoms, with persistence, within 4 hours. Persistence refers to the extent of relief wherein greater than or equal to 20 mm decrease in VAS score is achieved.

FIG. 3 is a chart depicting the number of recurrences of attack symptoms or emergence of new attack symptoms within 3 days following the rhC1INH treatment described in Example 1.

FIG. 4 is a graph depicting the baseline median D-dimer concentrations, which were elevated from normal levels for the HAE patients referenced in Example 2.

FIG. 5 is a graph depicting the change in median D-dimer concentrations over time for the patients on placebo vs. rhC1INH (Example 2) as compared with normal D-dimer concentration levels.

FIG. 6 is a graph depicting the D-dimer concentrations for patients on placebo who had submucosal or subcutaneous attack sites (Example 2) as compared with normal D-dimer concentration levels.

FIG. 7 is a graph depicting the D-dimer concentration in patients on rhC1INH who had submucosal or subcutaneous attack sites (Example 2) as compared with normal D-dimer concentration levels.

FIG. 8 is a graph depicting the D-dimer concentrations for patients with multiple vs. single attack sites (Example 2) as compared with normal D-dimer concentration levels.

FIG. 9 is a graph depicting the D-dimer concentration for patients administered rhC1INH vs. placebo (Example 2).

### DETAILED DESCRIPTION

Hereditary angioedema with C1 esterase inhibitor (ClINH) deficiency is characterized by recurrent attacks of tissue swelling. Recombinant human C1INH (rhC1INH) is effective in improving angioedema symptoms in HAE patients. The treatment results in a high response rate and no relapses within at least 12 hours, 24 hours, within 48 hours or for 72 hours. The treatment provides substantial relief or the beginning of relief within 4 hours. In a further embodiment, a single dose of rhC1INH provides sustained and durable responses in the treatment of acute HAE attacks. In another embodiment, the method includes administering to the patient a first dose and a second dose of recombinant C1 inhibitor is administered after the first dose, each dose at 50 IU/kg body weight of the patient. In methods described herein, treatment with the recombinant C1 inhibitor described herein does not result in side effects or present risk of side effects such as elevated D-dimer levels, thromboembolic events, or deep vein thrombosis.

One aspect described herein is a method for treating a patient suffering from hereditary angioedema (HAE) comprising administering to the patient a composition comprising a C1 inhibitor wherein substantial relief of symptoms is achieved within 4 hours or less. In another aspect described herein is a method for treating a patient suffering from an acute HAE attack comprising administering to the patient a composition comprising a C1 inhibitor wherein the treatment substantially relieves the patient of the symptoms from the acute HAE attack and there is substantially no recurrence of symptoms within 12 hours, 24 hours, 48 hours, or 72 hours. Moreover, there is preferably no new acute HAE attack within 12 hours, 24 hours, 48 hours, or 72 hours.

In another aspect described herein is a method for treating a patient suffering from HAE comprising administering to the patient a composition comprising a C1 inhibitor wherein the treatment provides substantial relief of HAE symptoms but does not substantially elevate the patient D-dimer level. In one embodiment, the D-dimer level is not substantially elevated over a period of at least 7 days from the administration of the C1 inhibitor composition. In another embodiment, the patient D-dimer level remains lower than 4000 ug/L, lower than 3000 ug/L, lower than 2500 ug/L, from the time of treatment through at least 7 days after the treatment.

In a further aspect described herein is a method for treating a patient suffering from a HAE comprising administering to the patient a composition comprising a C1 inhibitor wherein the treatment provides substantial relief of HAE symptoms but does not substantially increase the risk of a thromboembolic event. The treatment further does not substantially increase the risk of deep vein thrombosis.

For each of the above embodiments, the C1 inhibitor composition can be administered to a patient with one or more submucosal or subcutaneous locations of attack. In one embodiment, the C1 inhibitor is rhC1INH. In each of the above aspects described herein, the composition is administered as a single dose or multiple doses, preferably in a single dose. The composition is administered in a dosage of 25 to 100 IU/kg, more preferably at about 50 IU/kg, or at 50 IU/kg. The dosage is preferably administered intraveneously.

In the above embodiments, the C1 inhibitor composition is preferably administered to provide relief of symptoms from an HAE attack, i.e. to induce a substantial reduction in tissue swelling due to HAE.

### C1Inhibitor Genes

The C1 inhibitor cDNA sequence was shown to encode a protein of 500 amino acids, including a 22 amino acid signal sequence (Bock et al. 1986, Biochem. 25: 4292-4301). The entire human genomic sequence of C1 inhibitor is known and shows that the gene comprises 7 introns (Carter P. et al. 1988, Eur. J. Biochem. 173: 163). Transgenic mammals expressing allelic, cognate and induced variants of any of the prototypical sequence described in this reference are included in the invention. Such variants usually show substantial sequence identity at the amino acid level with known C1 inhibitor genes. Such variants usually hybridize to a known gene under stringent conditions or cross-react with antibodies to a polypeptide encoded by one of the known genes. Other examples of genomic and cDNA sequences are available from GenBank. To the extent that additional cloned sequences of C1 inhibitor genes are required, they may be obtained from genomic or cDNA libraries (preferably human) using known C1 inhibitor sequences.

### Transgene Design

Transgenes are designed to target expression of a recombinant C1 inhibitor to the mammary gland of a transgenic non-human mammal harboring the transgene. The basic approach entails operably linking an exogenous DNA segment encoding the protein with a signal sequence, and a regulatory sequence effective to promote expression of the exogenous DNA segment. Typically, the regulatory sequence includes a promoter and enhancer. The DNA segment can be genomic, minigene (genomic with one or more introns omitted), cDNA, a YAC fragment, a chimera of two different C1 inhibitor genes, or a hybrid of any of these. Inclusion of genomic sequences generally leads to higher levels of expression.

In genomic constructs, it is not necessary to retain all intronic sequences. For example, some intronic sequences can be removed to obtain a smaller transgene facilitating DNA manipulations and subsequent microinjection. See Archibald et al., WO 90/05188 (incorporated by reference in its entirety for all purposes). Removal of some introns is also useful in some instances to enhance expression levels. Removal of one or more introns to reduce expression levels to ensure that posttranslational modification is substantially complete may also be desirable. It is also possible to delete some or all of the non-coding exons. In some transgenes, selected nucleotides in C1 inhibitor encoding sequences are mutated to remove proteolytic cleavage sites. Because the intended use of C1 inhibitors produced by transgenic mammals is usually administration to humans, the species from which the DNA segment encoding a C1 inhibitor sequence is obtained is preferably human. Analogously if the intended use were in veterinary therapy (e.g., on a horse, dog or cat), it is preferable that the DNA segment be from the same species. Regulatory sequences such as a promoter and enhancer are from a gene that is exclusively or at least preferentially expressed in the mammary gland (e.g., a mammary-gland specific gene). Preferred genes as a source of promoter and enhancer include β-casein, κ-casein, αS1 -casein, αS2-casein, β-lactoglobulin, whey acid protein, and α-lactalbumin. The promoter and enhancer are usually but not always obtained from the same mammary-gland specific gene. Preferably this gene is from the same species of mammal as the mammal into which the transgene is to be expressed. Expression regulation sequences from other species such as those from human genes can also be used. The signal sequence must be capable of directing the secretion of the C1 inhibitor from the mammary gland. Suitable signal sequences can be derived from mammalian genes encoding a secreted protein. The natural signal sequences of C1 inhibitors are suitable. In addition to such signal sequences, preferred sources of signal sequences are the signal sequence from the same gene as the promoter and enhancer are obtained. Optionally, additional regulatory sequences are included in the transgene to optimize expression levels. Such sequences include 5′ flanking regions, 5′ transcribed but untranslated regions, intronic sequences, 3′ transcribed but untranslated regions, polyadenylation sites, and 3′ flanking regions. Such sequences are usually obtained either from the mammary-gland specific gene from which the promoter and enhancer are obtained or from the C1 inhibitor gene being expressed. Inclusion of such sequences produces a genetic milieu simulating that of an authentic mammary gland specific gene and/or that of an authentic C1 inhibitor gene. This genetic milieu results in some cases (e.g., bovine αS1-casein) in higher expression of the transcribed gene. Alternatively, 3′ flanking regions and untranslated regions are obtained from other heterologous genes such as the β-globin gene or viral genes. The inclusion of 3′ and 5′ untranslated regions from a C1 inhibitor gene, or other heterologous gene can also increase the stability of the transcript.

In some embodiments, about 0.5, 1, 5, 10, 15, 20 or 30 kb of 5′ flanking sequence is included from a mammary specific gene in combination with about 1, 5, 10, 15, 20 or 30 kb or 3′ flanking sequence from the C1 inhibitor gene being expressed. If the protein is expressed from a cDNA sequence, it is advantageous to include an intronic sequence between the promoter and the coding sequence. The intronic sequence is preferably a hybrid sequence formed from a 5′ portion from an intervening sequence from the first intron of the mammary gland specific region from which the promoter is obtained and a 3′ portion from an intervening sequence of an IgG intervening sequence or C1 inhibitor gene. See DeBoer et al., WO 91/08216 (incorporated by reference in its entirety for all purposes). Another preferred transgene for expressing a C1 inhibitor cDNA is based on the pBC1 expression vector kit, which is commercially available from Invitrogen (Carlsbad, Calif.). The pBC1 vector comprises the goat β-casein promoter as well as parts of the goat β-casein gene, which include several exons and introns, as well as 3′ untranslated sequences. Insertion of the C1 inhibitor cDNA into the unique Xho insertion site of pBC1 will produce a chimeric RNA comprising the C1 inhibitor cDNA sequences flanked by the goat β-casein exon and intron sequences. However, upon proper splicing of this chimeric RNA molecule, only the C1 inhibitor cDNA sequences is translated.

A preferred transgene for expressing a C1 inhibitor protein from genomic sequences comprises a genomic C1 inhibitor sequence encoding the entire coding sequence and a signal peptide, a 3′ UTR and a 3′ flanking sequence, operably linked to a 5′ alpha SI casein fragment containing regulatory sequence(s) sufficient to direct expression of the C1 inhibitor protein.

DNA sequence information is available for all of the mammary gland specific genes listed above, in at least one, and often several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985)) (rat β-casein); Yu-Lee & Rosen, J. Biol. Chem. 258, 10794-10804 (1983) (rat γ-casein)); Hall, Biochem. J. 242, 735-742 (1987) (α-lactalbumin human); Stewart, Nucleic Acids Res. 12, 389 (1984) (bovine αs1 and K casein cDNAs); Gorodetsky et al., Gene 66, 87-96 (1988) (bovine β casein); Alexander et al., Eur. J. Biochem. 178, 395-401 (1988) (bovine K casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin) (incorporated by reference in their entirety for all purposes).

The structure and function of the various milk protein genes are reviewed by Mercier & Vilotte, J. Dairy Sci. 76, 3079-3098 (1993) (incorporated by reference in its entirety for all purposes). To the extent that additional sequence data might be required, sequences flanking the regions already obtained could be readily cloned using the existing sequences as probes. Mammary-gland specific regulatory sequences from different organisms are likewise obtained by screening libraries from such organisms using known cognate nucleotide sequences, or antibodies to cognate proteins as probes.

General strategies and exemplary transgenes employing αS1-casein regulatory sequences for targeting the expression of a recombinant protein to the mammary gland are described in more detail in DeBoer et al., WO 91/08216 and WO 93/25567 (incorporated by reference in their entirety for all purposes). Examples of transgenes employing regulatory sequences from other mammary gland specific genes have also been described. See, e.g., Simon et al., Bio/Technology 6, 179-183 (1988) and WO 88/00239 (1988) (β-lactoglobulin regulatory sequence for expression in sheep); Rosen, EP 279,582 and Lee et al., Nucleic Acids Res. 16, 1027-1041 (1988) (β-casein regulatory sequence for expression in mice); Gordon, Biotechnology 5, 1183 (1987) (WAP regulatory sequence for expression in mice); WO 88/01648 (1988) and Eur. J. Biochem. 186, 43-48 (1989) (α-lactalbumin regulatory sequence for expression in mice) (incorporated by reference in their entirety for all purposes).

The transgenes described above are introduced into non-human mammals. Most non-human mammals, including rodents such as mice and rats, rabbits, ovines such as sheep, caprines such as goats, porcines such as pigs, and bovines such as cattle and buffalo, are suitable. Bovines offer an advantage of large yields of milk, whereas mice offer advantages of ease of transgenesis and breeding. Rabbits offer a good compromise of these advantages. A rabbit can yield 100 ml milk per day with a protein content of about 14% (see Buhler et al., Bio/Technology 8, 140 (1990)) (incorporated by reference in its entirety for all purposes), and yet can be manipulated and bred using the same principles and with similar facility as mice. Nonviviparous mammals such as a spiny anteater or duckbill platypus are typically not employed.

In some methods of transgenesis, transgenes are introduced into the pronuclei of fertilized oocytes. For some animals, such as mice and rabbits, fertilization is performed in vivo and fertilized ova are surgically removed. In other animals, particularly bovines, it is preferable to remove ova from live or slaughterhouse animals and fertilize the ova in vitro. See DeBoer et al., WO 91/08216. In vitro fertilization permits a transgene to be introduced into substantially synchronous cells at an optimal phase of the cell cycle for integration (not later than S-phase). Transgenes are usually introduced by microinjection. See U.S. Pat. No. 4,873,292. Fertilized oocytes are then cultured in vitro until a pre-implantation embryo is obtained containing about 16-150 cells. The 16-32 cell stage of an embryo is described as a morula. Pre- implantation embryos containing more than 32 cells are termed blastocysts. These embryos show the development of a blastocoele cavity, typically at the 64-cell stage. Methods for culturing fertilized oocytes to the pre-implantation stage are described by Gordon et al., Methods Enzymol. 101, 414 (1984); Hogan et al., Manipulation of the Mouse Embryo: A Laboratory Manual, C.S.H.L. N.Y. (1986) (mouse embryo); Hammer et al., Nature 315, 680 (1985) (rabbit and porcine embryos); Gandolfi et al. J. Reprod. Fert. 81, 23-28 (1987); Rexroad et al., J. Anim. Sci. 66, 947-953 (1988) (ovine embryos) and Eyestone et al. J. Reprod. Fert. 85, 715-720 (1989); Camous et al., J. Reprod. Fert. 72, 779-785 (1984); and Heyman et al. Theriogenology 27, 5968 (1987) (bovine embryos) (incorporated by reference in their entirety for all purposes). Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, transgenes can be introduced into embryonic stem cells (ES). These cells are obtained from preimplantation embryos cultured in vitro. Bradley et al., Nature 309, 255-258 (1984) (incorporated by reference in its entirety for all purposes). Transgenes can be introduced into such cells by electroporation or microinjection. ES cells are suitable for introducing transgenes at specific chromosomal locations via homologous recombination. For example, a transgene encoding C1 inhibitor can be introduced at a genomic location at which it becomes operably linked to an endogenous regulatory sequence that can directed expression of the coding sequence in the mammary gland. Transformed ES cells are combined with blastocysts from a non-human animal. The ES cells colonize the embryo and in some embryos form or contribute to the germline of the resulting chimeric animal. See Jaenisch, Science, 240, 1468-1474 (1988) (incorporated by reference in its entirety for all purposes). Alternatively, ES cells can be used as a source of nuclei for transplantation into an enucleated fertilized oocyte, giving rise to a transgenic mammal. In a further embodiment, transgenic animals, preferably non-human mammals, containing a transgenes capable of expressing C1 inhibitor are produced by methods involving nuclear transfer. Various types of cells can be employed as donors for nuclei to be transferred into oocytes. Donor cells can be obtained from all tissues of transgenic animals containing a C1 inhibitor transgenes, such as adult, fetal or embryonic cells, at various stages of differentiation, ranging from undifferentiated to fully differentiated, in various cell cycle stages, e.g. both quiescent and proliferating cells, and obtained form either somatic or germline tissues (see WO 97/07669, WO 98/30683 and WO 98/39416. each incorporated by reference in their entirety for all purposes).

Alternatively, donor nuclei are obtained from cells cultured in vitro into which a C1 inhibitor transgene is introduced using conventional methods such as Ca-phosphate transfection, microinjection or lipofection and which have subsequently been selected or screened for the presence of a transgene or a specific integration of a transgene (see WO 98/37183 and WO 98/39416, each incorporated by reference in their entirety for all purposes). Donor nuclei are introduced into oocytes by means of fusion, induced electrically or chemically (see any one of WO 97/07669, WO 98/30683 and WO 98/39416), or by microinjection (see WO 99/37143, incorporated by reference in its entirety for all purposes). Transplanted oocytes are subsequently cultured to develop into embryos which are subsequently implanted in the oviducts of pseudopregnant female animals, resulting in birth of transgenic offspring (see any one of WO 97/07669, WO 98/30683 and WO 98/39416).

Another method of transgenesis uses (retro)virus-based vectors to introduce the desired transgenes. Examples of such vectors include the vesicular stomatitis virus G glycoprotein (VSG-G) MoMLV derived retroviral vector (VSV-G pseudotype) as described by Yee et al. (1994, Meth. Cell. Biol. 43: 99-112, incorporated by reference in its entirety for all purposes). Non-human mammalian, preferably bovine, oocytes arrested in metaphase II of the second meiotic division before fertilization are infected with such a VSV-G pseudotype vector as described by Chan et al (1998, Proc. Natl. Acad. Sci. USA 95: 14028-14033, incorporated by reference in its entirety for all purposes) to produce transgenic offspring. Alternatively, instead of producing a genetically modified animal, a restricted organ, preferably a mammary gland is transformed by retroviral infection for the purpose of making pharmaceutical proteins. Infusion retroviral vectors, carrying sequences encoding C1 inhibitor, into non-human mammary glands to infect the mammary epithelial cells allow the production of the C1 inhibitor protein in the milk of these animals (Archer et al., 1994, Proc. Natl. Acad. Sci. USA 91:6840-6844, incorporated by reference in its entirety for all purposes).

For production of transgenic animals containing two or more transgenes, the transgenes can be introduced simultaneously using the same procedure as for a single transgene. Alternatively, the transgenes can be initially introduced into separate animals and then combined into the same genome by breeding the animals. Alternatively, a first transgenic animal is produced containing one of the transgenes. A second transgene is then introduced into fertilized ova or embryonic stem cells from that animal. In some embodiments, transgenes whose length would otherwise exceed about 50 kb, are constructed as overlapping fragments. Such overlapping fragments are introduced into a fertilized oocyte or embryonic stem cell simultaneously and undergo homologous recombination in vivo. See Kay et al., WO 92/03917 (incorporated by reference in its entirety for all purposes).

Transgenic mammals described herein incorporate at least one transgene in their genome as described above. Introduction of a transgene at the one cell stage usually results in transgenic animals and their progeny substantially all of whose germline and somatic cells (with the possible exception of a few cells that have undergone somatic mutations) contain the transgene in their genomes. Introduction of a transgene at a later stage leads to mosaic or chimeric animals. However, some such animals that have incorporated a transgene into their germline can be bred to produce transgenics in which substantially all of whose somatic and germline cells contain a transgene. Viral transgenesis of mammary gland cells usually results in a transgenic mammal in which the transgene is present only in mammary gland cells. Such a mammal does not transmit its germline to future generations.

The transgene targets expression of a DNA segment encoding a C1 inhibitor protein at least predominantly to the mammary gland. C1 inhibitor can be secreted at high levels of at least 100, 500, 1000, 2000, 5000 or 10,000, 20,000 or 50,000 μg/ml. The transgenic mammals described herein exhibit substantially normal health. Secondary expression of C1 inhibitor proteins in tissues other than the mammary gland does not occur to an extent sufficient to cause deleterious effects. Moreover, exogenous C1 inhibitor protein is secreted from the mammary gland with sufficient efficiency that no problem is presented by deposits clogging the secretory apparatus.

The age at which transgenic mammals can begin producing milk, of course, varies with the nature of the animal. For transgenic bovines, the age is about two-and-a-half years naturally or six months with hormonal stimulation, whereas for transgenic mice the age is about 9-11 weeks. Of course, only the female members of a species are useful for producing milk. However, transgenic males are also of value for breeding female descendants. The sperm from transgenic males can be stored frozen for subsequent in vitro fertilization and generation of female offspring. F. Recovery of Proteins from Milk or Other Sources Transgenic adult female mammals produce milk containing high concentrations of exogenous C1 inhibitor protein.

Purification of C1 inhibitor from milk can be carried out by defatting of the transgenic milk by centrifugation and removal of the fat, followed by removal of casein's by high speed centrifugation followed by dead-end filtration (e.g., dead-end filtration by using successively declining filter sizes) or cross-flow filtration, or; removal of casinos directly by cross filtration. The protein can be purified from milk, if desired, by virtue of its distinguishing physical and chemical properties (see generally Scopes, Protein Purification (Springer-Verlag, N.Y., 1982)) Prograis et al., (1985) J. Medicine 16 (1-3): 303-350; Pilatte et al, (1989) J. Immunol. Methods 120: 37-43, Reboul et al., (1977) Febs Lett. 79: 45-50, Alsenz et al., (1987) J. Immunol. Methods 96: 107-114, Ishizaki et al., (1977) J. Biochem. 82: 1155-1160. The conditions of purification should preferably separate human C1 inhibitor from endogenous C1 inhibitor of the nonhuman transgenic mammal.

Cationic, anionic and metal-affinity chromatography can all be used for purification of human C1 inhibitor protein, from milk or other sources, such as recombinant cell cultures or natural sources. Some methods use more than one of these steps, and some methods use all three steps. Although the steps can be performed in any order, a preferred order is to perform cationic chromatography, followed by anionic chromatography, followed by metal ion affinity chromatography.

Cationic chromatography can be performed, for example, using Sepharose™ big beads or carboxymethyl-cellulose. A low salt loading buffer (e.g., 20 mM sodium citrate, 0.02 M sodium chloride) is used. Human C1 inhibitor can be eluted at higher salt concentration (e.g., 20 mM sodium citrate, 0.2 M sodium chloride). Eluate containing human C1 inhibitor is then subject to anionic chromatography. The matrix of an anionic column can be a material such as cellulose, dextrans, agarose or polystyrene. The ligand can be eithylaminoethyl (DEAE), polyethyleneimine (PEI) or a quaternary ammonium functional group example. The strength of an anion exchange column refers to the state of ionization of the ligand. Strong ionic exchange columns, such as those having a quaternary ammonium ligand, bear a permanent positive charge. In weak anion exchange columns, such as DEAE and PEI, the existence of the positive charge depends on the pH of the column. Anion exchange columns are generally loaded with a low-salt buffer at a pH above the pi of human C1 inhibitor. The columns are washed several times in the low-salt buffer to elute proteins that do not bind. Proteins that have bound are then eluted using a buffer of increased salt concentration.

Q Sepharose FF is a preferred anion exchange column because this material is relatively inexpensive compared with other anion-exchange columns and has a relatively large bead size suitable for large scale purification. Under specified conditions, human C1 inhibitor can be eluted from Q Sepharose FF without eluting rabbit C1 inhibitor or other proteins found in rabbit milk. To obtain good binding of human acid α-glucosidase to the Q Sepharose FF, the column is pre-equilibrated in low salt (e.g., less than 50 mM, such as sodium phosphate buffer. The pH of the buffer should be about 7.0+/−1.0 to obtain a good binding of human C1 inhibitor to the column. Human C1 inhibitor is then eluted by step-wise or gradient elution at increased salt concentration. Step-wise elution is more amenable to large-scale purification. Most loaded human C1 inhibitor can be eluted from a Q FF column in one step (at about 0.25 M salt) with relatively high purity.

Metal affinity chromatography is conducted using a matrix, such as Sepharose, and a bound metal ion, such as copper, zinc, nichol, cobalt or calcium. Organic chelating groups such as iminodiacetic acid can also be used. The column is equilibrated at a pH of about 6-8 with a nonchelating salt (e.g., sodium chloride) present at a relatively high concentration e.g., greater than 0.2 M. Under these conditions, residual contaminating proteins bind to the column, whereas human C1 inhibitor does not, and can be readily eluted.

An exemplary purification procedure is described in the Examples section. This procedure provides a C1 inhibitor preparation, which is at least 98% or 99% o pure (w/w) with respect to all contaminants and contains less than 0.5%, 0.1% or 0.05% rabbit C1 inhibitor (w/w). Additional purification are preferably used to obtain C1 inhibitor preparations with a purity of at least 99%), preferably at least 99.5%, more preferably 99.8% and most preferably 99.9%.

### Pharmaceutical Compositions

In some methods, C1 inhibitor purified from milk or other source is administered in purified form together with a pharmaceutical carrier as a pharmaceutical composition. The preferred form depends on the intended mode of administration and therapeutic application. The pharmaceutical carrier can be any compatible, nontoxic substance suitable to deliver the polypeptides to the patient. Sterile water, alcohol, fats, waxes, and inert solids may be used as the carrier. Pharmaceutically acceptable adjuvants, buffering agents, dispersing agents, and the like, may also be incorporated into the pharmaceutical compositions. The concentration of the inhibitor in the pharmaceutical composition can vary widely, e.g., from less than about 0.1% by weight, usually being at least about 1% by weight to as much as 20% by weight or more.

The pharmaceutical composition is preferably administered by parenteral administration, such as for example by intravenous, intra-arterial, subcutaneous, intraperitoneal or intramuscular injection or infusion; or by intrathecal or intracranial administration. In a preferred embodiment it is administered by intravenous infusion. Suitable formulations for parenteral administration are known in the art and are typically liquid formulations.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

Data from patients (13 years or older) with laboratory confirmed diagnosis of HAE treated with intravenous rhC1INH (50 IU/kg) were pooled from two randomized controlled trials and their open-labeled extension studies. Patients were observed for onset of symptoms less than 5 hours before presentation and a baseline visual analog scale (VAS; Reidl M A, Ann Allergy Asthma Immunol 2013, 110(4):295-9, which is incorporated herein by reference) score of greater than or equal to 50 mm (severe). In one of the randomized control trials, patients could receive a second rhC1INH dose as rescue medication for life-threatening symptoms or if no relief occurred by 4 hours after the first dose. In the other randomized controlled trial, the patients were not permitted a second dose. In the open-labeled extension studies, second doses of rhC 1INH were permitted based on clinical responses at the discretion of investigators.

127 patents received rhC1INH 50 IU/kg for one or more attacks in the course of the 4 clinical trials. 121 patients were eligible to receive a second dose of rhC1INH. Response, relapse and recurrence data were combined for all attacks at all anatomical sites. Response was defined as relief within 4 hours of treatment with persistence (greater than or equal to 20 mm decrease in VAS scores as two consecutive time points) within 4 hours and no additional dose or rescue medication before persistence. Relapse was determined for all patients with 24 hour follow-up data and recurrence or new attack symptoms were determined for all patients with 3-day follow-up data.

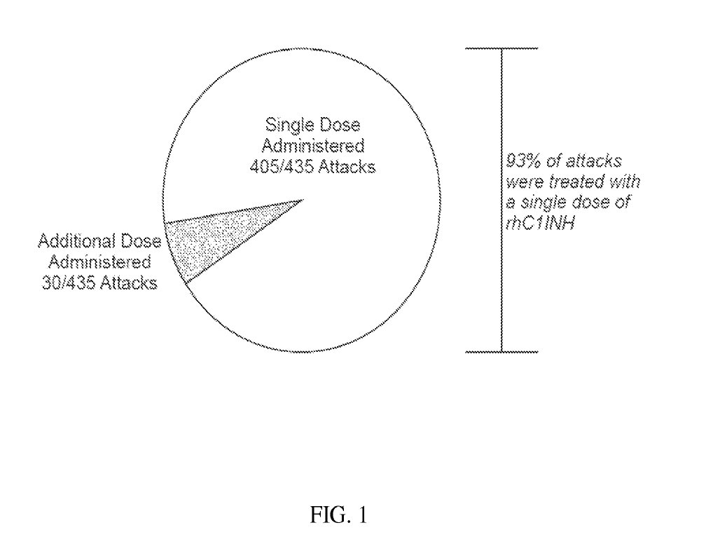

FIG. 1 shows the number of rhC1INH doses administered for acute HAE attacks. 93% of attacks were treated with a single dose of rhC1INH. 7% of attacks were treated with two doses of rhC1INH.

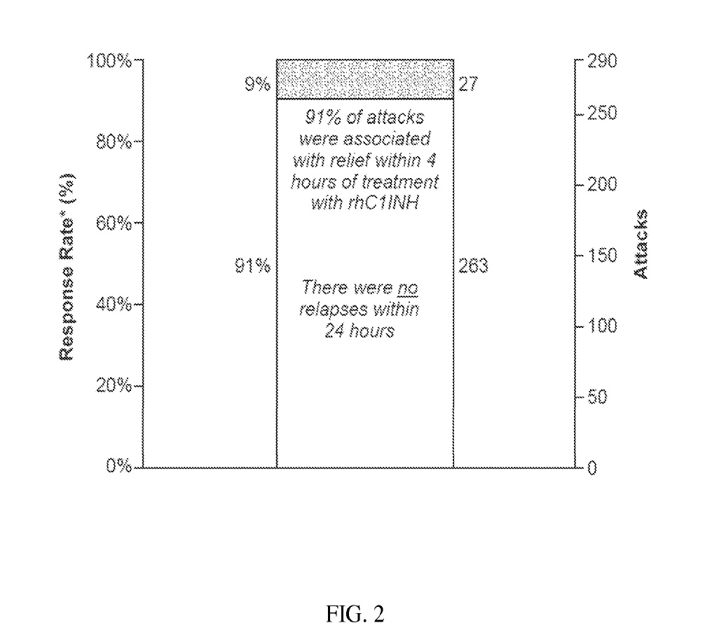

FIG. 2 shows response and relapse rates in rhC1INH-treated patients. A response was defined as beginning of relief of symptoms, with persistence, within 4 hours. For patients in the two randomized controlled trials and two open-label extension studies, no thrombotic or thromboembolic events, no anaphylactic reactions, and no induction on neutralizing antibodies following treatment with rhC1INH.

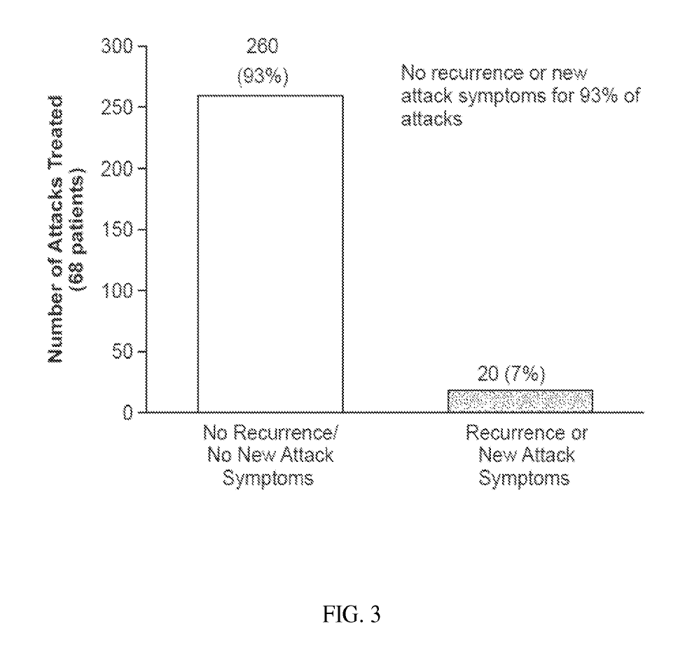

FIG. 3 shows the number of recurrences of attack symptoms or new attack symptoms within 3 days following rhC1INH treatment. In 93% of patents, there was no recurrence or new attack symptoms.

Based on this study, it was found that treatment with rhC1INH resulted in a high response rate, assessed as the number of attacks with beginning of relief within 4 hours. Most attacks with beginning of relief within 4 hours were treated effectively with a single rhC1INH dose. Some attacks required a second rhC1INH dose. No significant relapses occurred within 24 hours for attacks with relief within 4 hours. Incidence of recurrent or new attack symptoms within 3 days of rhC1NH treatment was low. Further, a single dosage of rhC1NH provided sustained and durable responses in the treatment of acute HAE attacks.

### Example 2

Thromboembolic events (TEE) have been reported with some plasma-derived C1INH, but not with recombinant human C1INH (rhC1INH; greater than 1000 administrations). This study evaluated safety and efficacy of rhC1INH for acute HAE attacks included monitoring for TEE and assessments of D-dimer fibrin-degradation products (D-dimer levels) and risk of deep vein thrombosis (DVT).

Seventy-four patients with acute HAE attacks were randomized 3:2 and received 50 IU/kg rhC1INH or placebo. D-dimer levels (presented as median 25^{th}-75^{th} quartiles) were assessed prior to, and 2 hours and at day 7 after study drug infusion. DVT risk was assessed using Wells Prediction Rule. Wells P S, et al. Thromb. Haemost. 2000:83:416-20. D-dimer levels were evaluated by blood samples collected at baseline (e.g., less than 5 hours from onset and prior to study medication), at 2 hours and at day 7 (after the attack resolved) following intravenous injection of study medication. Values less than or equal to 250 μg/L were considered normal (e.g., reference standard).

Patients and study design: This was a randomized, double-blind, placebo (saline)-controlled, multicenter, multinational study to evaluate the efficacy and safety of rhC1INH compared with saline, for the treatment of acute angioedema attacks in patients with HAE. Seventy-five patients (age ≧13 years; ≧18 years outside the United States and Canada), with a laboratory-confirmed diagnosis of HAE, were randomized centrally (3:2) to receive a double-blind, intravenous injection of rhC1INH (50 IU/kg for patients <84 kg, or 4200 IU for patients≧84 kg) or saline for treatment of an eligible angioedema attack. Patients were eligible for treatment if (i) the location of their attack was peripheral (extremities), abdominal, facial, and/or oropharyngeal-laryngeal; (ii) the onset of these attacks was less than 5 hours prior to presentation to the clinic; and (iii) the overall severity of the attack was rated by the patient to be at least 50 mm on a Visual Analog Scale (VAS) of 100 mm (Reidl M A, Ann Allergy Asthma Immunol 2013, 110(4):295-9), which is incorporated herein by reference). For patients with multiple eligible attack locations, the primary attack location was defined as the location with the highest VAS score at baseline.

Thrombotic Risk Assessments: All randomized patients were clinically monitored for thrombotic events. The risk of deep vein thrombosis (DVT) was assessed by using the Wells prediction rule (Wells P S, et al. Thromb. Haemost. 2000:83:416-20); patients with elevated scores post-dose were required to have an extremity ultrasound to rule out DVT. Patients were evaluated for post-infusion increase in D-dimer levels for the possible development of thrombotic events (including ultrasound if clinically indicated).

Plasma Sample Collection: For determination of D-dimer levels, citrated blood samples were collected at baseline (e.g., less than five hours from onset and prior to intravenous injection of study medication), at two hours and at Day 7 (after the attack resolved) following intravenous injection of study medication. For all analyses, patients randomized to receive saline solution who also received rhC1INH as a rescue medication were switched from the saline solution treatment group to the rhC1INH treatment group for any assessments after the receipt of rescue medication.

D-Dimer Measurement: D-dimer levels in the plasma were measured in a central laboratory (normal range <540 μg/L).

Patient demographics: Seventy-five patients presenting with eligible acute HAE attacks were enrolled to receive study medication: 44 were randomized to 50 IU/kg rhC1INH and 31 were randomized to saline; one patient randomized to rhC1HNH treatment was not treated and not included in the analyses.

Patient disposition, key demographics, and HAE attack frequency and severity of the eligible attack are summarized by treatment group in Table 1. Patient demographics and baseline characteristics were generally similar between the treatment groups. Attack severity at baseline, as rated by the patients using a 100 mm VAS scale, was similar in both groups (average for the rhC1INH group 73.5 mm vs 77.3 mm for the saline group). The primary attack locations were also similar in the rhC1INH and the saline groups (peripheral location in 44% of the rhC1INH vs 45% of the saline group, and an abdominal location in 37% of the rhC1INH group vs 39% of the saline group).

Risk of Deep Vein Thrombosis: None of the patients were identified as having an increased risk for DVT based on Wells prediction rule scores. All scores recorded in 39 patients in the rhC1INH group and 30 patients in the saline group, were low, ranging from −2 to 0, suggesting that the patients had a very low probability for having a DVT. Ultrasounds performed on two patients (1 rhC1INH and 1 saline) with Wells scores of 0 were normal in both abdomen and lower extremities with no evidence of DVT.

D-Dimer levels: D-dimer levels (presented as median [25^{th}-75^{th} quartiles]) were assessed at three time points (baseline, two hours following rhC1INH infusion, and seven days after treatment with rhC1INH). Further classification was done by assessing primary attack location type (submucosal: abdominal and oropharyngeal-laryngeal vs. subcutaneous: facial and peripheral), by severity (moderate: VAS between 50 and 75 mm; severe ≧76 mm for the primary attack location) and by single vs. multiple affected locations.

Overall median D-dimer levels were elevated in the patients at baseline (2149 [480-5105] μg/L, normal range <540 μg/L). (Table 2). D-dimer levels had continued to increase in all patients two hours after treatment with either rhC1INH or saline, to a median level of 2469 (643-5827) μg/L. By Day 7 post-treatment, D-dimer levels in both treatment groups were restored to near-normal levels. It should be noted that median D-dimer levels were not statistically different between the groups at two hours and Day 7 after treatment with either rhC1INH or saline. Mean changes from baseline in both treatment groups also were similar at two hours (rhC1INH: 145 μg/L; Saline: 192 μg/L) and Day 7 (rhC1INH: −2401 μg/L; saline: −1923 μg/L) in the two treatment groups suggesting that treatment by rhC1INH did not influence D-dimer production in HAE patients.

HAE attacks present as either submucosal or subcutaneous edema affecting the skin, intestines, and upper airway. D-dimer levels were evaluated in the patient population based on submucosal and subcutaneous primary attack locations (Table 3). Median D-dimer levels were at least three-fold higher than at baseline (p=0.0274) and two hours post-treatment (p=0.0126) in patients with submucosal attacks compared to patients with subcutaneous attacks. As in the overall population, treatment with rhC1INH had no apparent impact on the D-dimer levels at both post-dose time points. Comparisons between D-dimer levels at the individual primary attack locations (e.g., facial, peripheral, abdominal, oropharyngeal-laryngeal) were not further evaluated.

Severity at the primary attack location was classified as either moderate (VAS≧50 mm and <75 mm), or severe (VAS≧75 mm) at baseline. Overall, median baseline D-dimer levels were similar in patients with moderate (1674 [593-5241] μg/L) and severe (2320 [260-5550] μg/L) attacks (Table 4). Severe attacks treated with rhC1INH did tend to have lower D-dimer values (280 [109-925] μg/L) by Day 7 than those treated with saline (560 [273-4056]μg/L).

Although most HAE attacks present with symptoms isolated to a single location, some attacks may present with multiple anatomical locations affected at the same time. In light of this, it was also determined whether D-dimer levels were affected by the presence of multiple affected locations. Sixty-four patients reported single site attacks and ten reported multiple site attacks. At baseline, median D-dimer levels were higher in patients with multiple affected locations (9555 [4315-13300) μg/L) than in patients with single locations (4568 [2065-24634] μg/L). Two-hours after treatment, D-dimer levels were still more elevated with multiple attack locations (5040 [812-11045] μg/L vs. 2294 [615-5065] μg/L for single locations. By Day 7, D-dimer levels had returned to normal for both groups.

TABLE 1
Patient demographics and baseline characteristics

| | rhC1INH | | Saline | |
|---|---|---|---|---|
| | (N = 43) | | (N = 31) | |
| Female (%) | 64 | | 61 | |
| Caucasian (%) | 95 | | 97 | |
| Age at screening (yr) | | | | |
| Mean (SD) | 39.4 | (12.6) | 41.4 | (15.4) |
| Range | 17-67 | | 18-69 | |
| HAE attacks/y | | | | |
| Mean (SD) | 24.9 | (23.7) | 30.6 | (27.2) |
| Range | 0-143 | | 3-111 | |
| Use of prophylactic maintenance | N | (50) | N | (48) |
| therapy (n [%]) | | | | |
| Primary attack location (n [%])^{a} | | | | |
| Peripheral | 19 | (44) | 14 | (45) |
| Abdominal | 16 | (37) | 12 | (39) |
| Facial | 6 | (14) | 2 | (6) |
| Oropharyngeal-laryngeal | 2 | (5) | 3 | (10) |
| Overall severity VAS score at baseline | | | | |
| for primary attack location (mm) | | | | |
| Mean (SD) | 73.5 | (14.1) | 77.3 | (12. 6) |

| | | |
|---|---|---|
| Range | 50-100 | 49-100 |
| N | 43^{b} | 31 |

Abbreviation: VAS = Visual Analog Scale
^{a}For patients with >1 eligible attack location, the primary attack location was defined as the eligible location with the highest Overall Severity VAS score at baseline.
^{b}One patient (randomized to rhC1INH) did not receive study medication and is not included in the summary table.

TABLE 2
D-dimer levels* over time in all patients

| | Total |
|---|---|
| Time point | (N = 74) |
| Acute attack, Baseline, μg/L | |
| Mean (SD) | 4211 (5622) |
| Median | 2149 |
| Range | 6-24634 |
| n | 64 |
| 2 hours after treatment, μg/L | |
| Mean (SD) | 4421 (5740) |
| Median | 2469 |
| Range | 9-5827 |
| n | 68 |
| Day 7 after treatment, μg/L | |
| Mean (SD) | 1842 (2867) |
| Median | 425 |
| Range | 1-14250 |
| n | 64 |

*normal range < 540 μg/L

TABLE 3
D-dimer levels in symptomatic HAE patients with submucosal
vs. subcutaneous locations of the eligible attack.

| Time point/Anatomical | rhC1INH | Saline |
|---|---|---|
| Location* | (N = 43) | (N = 31) |
| Baseline, μg/L | | |
| Submucosal^{a} | 3095 (250-8676) | 3055 (1700-11350) |
| Subcutaneous^{b} | 1000 (500-4060) | 899 (260-3800) |
| 2 hours, μg/L | | |
| Submucosal | 4100 (1030-7731) | 5470 (2550-12500) |
| Subcutaneous | 1080 (730-4260) | 835 (310-2200) |
| Day 7, μg/L | | |
| Submucosal | 768 (266-4250) | 418 (245-2614) |
| Subcutaneous | 376 (150-1400) | 453 (246-2318) |

Values are presented as median (interquartile range).
*Anatomical location represents the primary attack location (see Methods)
^{a}Submucosal = Oropharyngeal-laryngeal, abdominal. No urogenital attacks were reported.
^{b}Subcutaneous = Peripheral, facial.

TABLE 4
D-dimer levels by severity at the primary attack location

| | Moderate | Severe |
|---|---|---|
| | (≧50 mm, <75 mm)^{a} | (≧75 mm)^{a} |
| Baseline, μg/L | 1674 (593-5241) | 2320 (260-5550) |
| 2 hours, μg/L | 2000 (656-5884) | 2678 (615-5840) |
| Day 7, μg/L | 1025 (382-3770) | 30 (150-1250) |

^{a}Severity is based on the overall VAS score at each visit at the primary attack location.

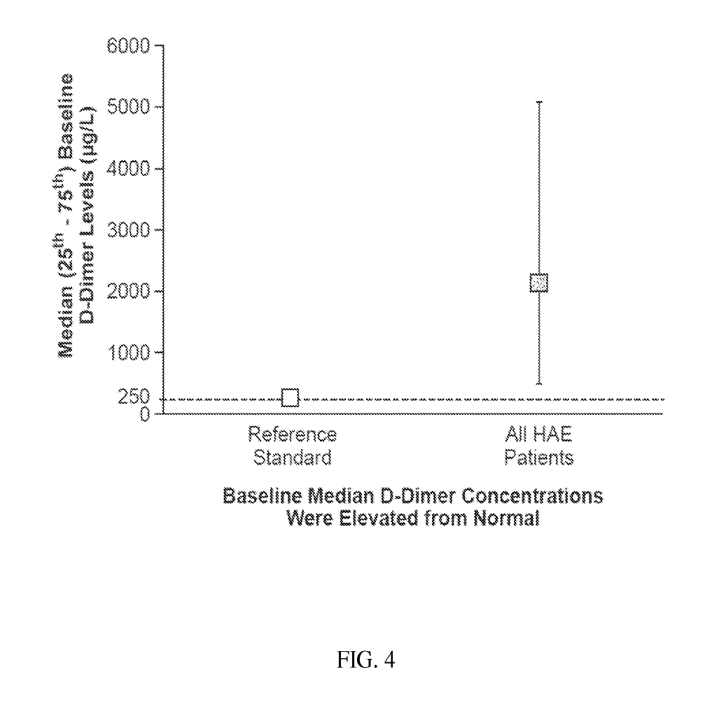

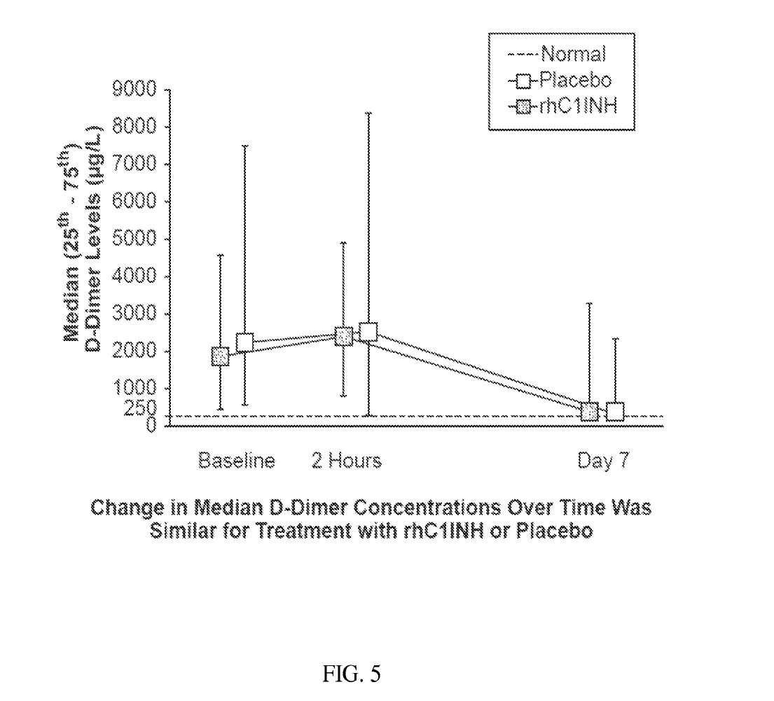

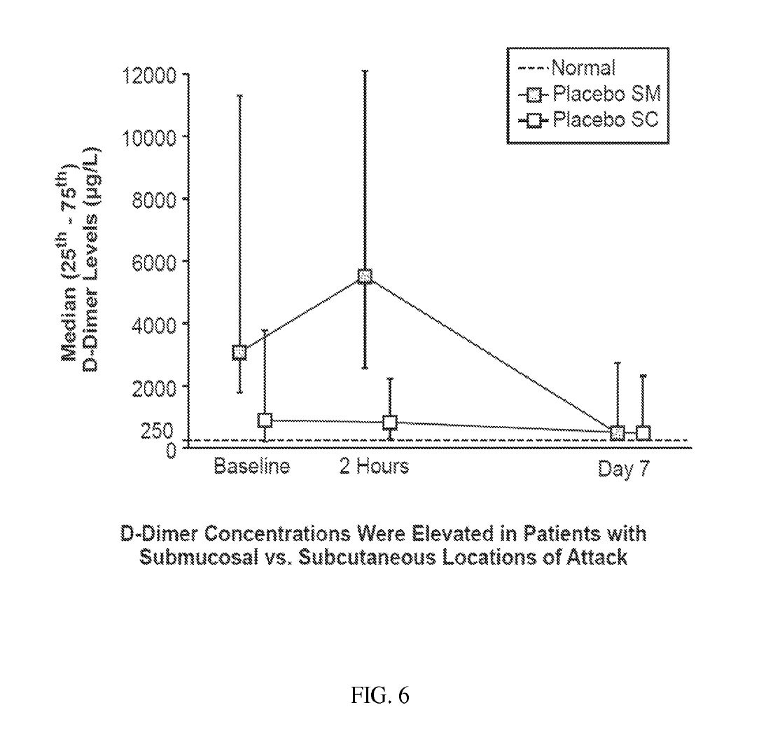

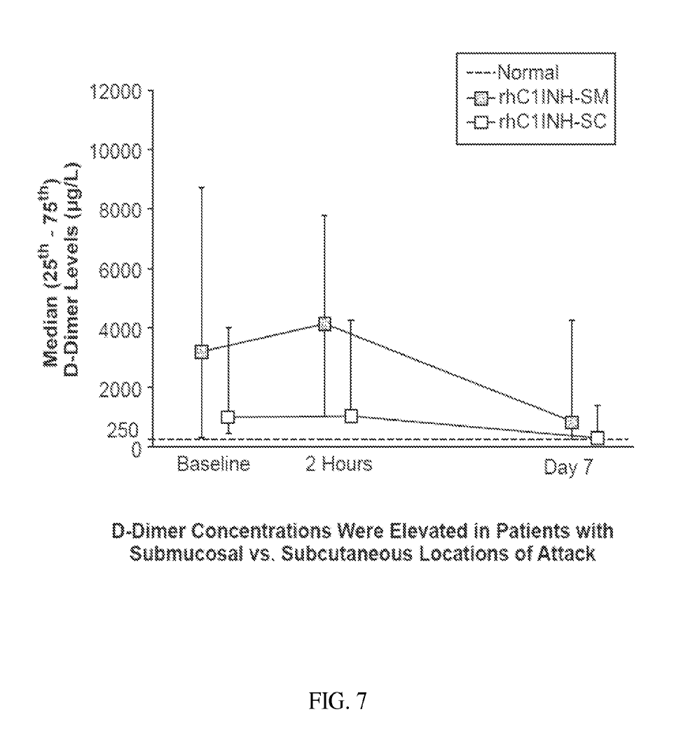

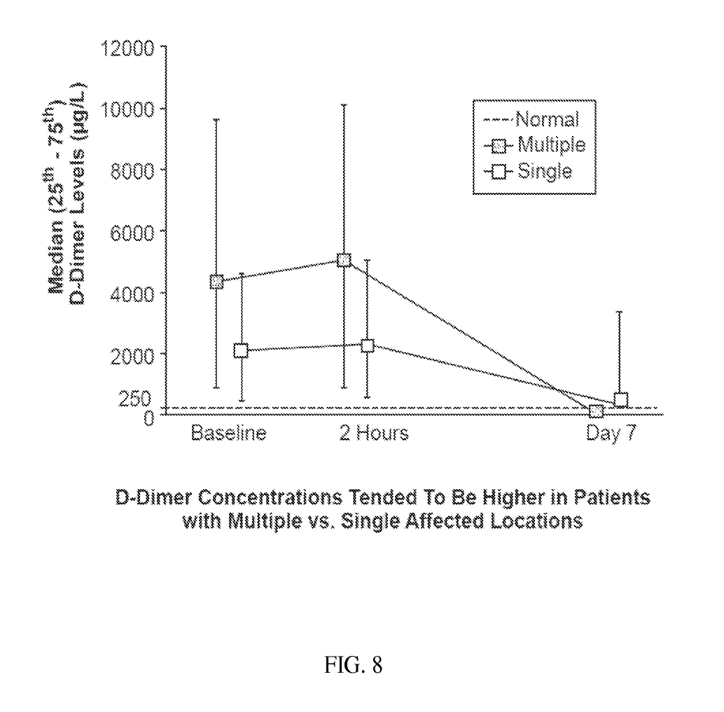

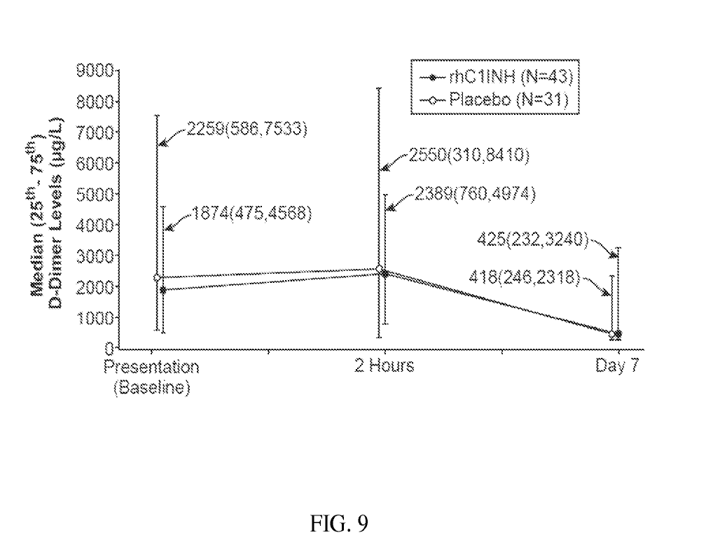

Results are further summarized in FIGS. 4-9. D-dimer levels were elevated during HAE attacks as compared with times of remission. However, elevation of D-dimer levels was not associated with rhC1INH treatment. No thromboembolic events were observed with rhC1INH.

The contents of all references, patents, pending patent applications and published patents cited throughout this application are hereby expressly incorporated by reference. Unless otherwise noted, the technical terms used herein are according to conventional usage as understood by persons skilled in the art. Definitions of common terms in molecular biology may be found in standard texts (e.g. Benjamin Lewin, Genes V, published by Oxford University Press, 1994 (ISBN 0-19854287-9); Kendrew et al. (eds.), The Encyclopedia of Molecular Biology, published by Blackwell Science Ltd, 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8)).

1. A method for treating an acute attack of hereditary angioedema (HAE) in a patient, said method comprising:
administering intravenously to the patient a first dose of a recombinant C1 esterase inhibitor at 50 IU/kg body weight of the patient; and
administering intravenously to the patient a second dose of the recombinant C1 esterase inhibitor at 50 IU/kg body weight of the patient after administration of the first dose, thereby treating the acute attack of HAE in the patient.

2. The method of claim 1, wherein the first dose is administered within five hours from onset of the attack of HAE in the patient.

3. The method of claim 1, wherein the second dose is administered at least four hours after the first dose.

4. The method of claim 1, wherein the first dose and the second dose are administered within a 24 hour period.

5. The method claim 1, wherein no more than two doses are administered within a 24 hour period.

6. The method of claim 1, wherein the patient has multiple attack sites.

7. The method of claim 1, wherein the attack site is peripheral, abdominal, facial, oropharyngeal, or laryngeal.

8. The method of claim 7, wherein the attack site is peripheral.

9. The method of claim 7, wherein the attack site is abdominal.

10. The method of claim 7, wherein the attack site is facial.

11. The method of claim 7, wherein the attack site is oropharyngeal.

12. The method of claim 7, wherein the attack site is laryngeal.

13. The method of claim 1, wherein the patient has life-threatening symptoms associated with the attack.

14. The method of claim 1, wherein the attack as a severity rating of at least 50 mm on a Visual Analog Scale (VAS) of 100 mm.

15. The method of claim 1, wherein the patient is an individual in whom the beginning of relief of symptoms occurs within 4 hours from the first dose and the extent of the relief is less than 20 mm decrease in VAS score prior to the second dose and/or wherein the decrease in VAS score is measured based on two consecutive time points.

16. The method of claim 1, wherein the patient is an individual in whom attack symptoms persist after the first dose.

17. The method of claim 1, wherein the recombinant C1 inhibitor has an amino acid sequence identical to the amino acid sequence of human plasma-derived C1 esterase inhibitor and a modified carbohydrate structure as compared to the human plasma-derived C1 esterase inhibitor.

18. The method of claim 1, wherein the recombinant C1 inhibitor is purified from the milk of transgenic rabbits.

19. The method of claim 1, wherein the recombinant C1 inhibitor is rhC1INH.

20. The method of claim 1, wherein the first dose and second dose are self-administered by the patient.
